(19) United States
(12) Patent Application Publication
(10) Pub. No.: US 2012/0324626 A1
(43) Pub. Date: Dec. 27, 2012

(54) EAR MUFF

(75) Inventor: Gabriele TORREGGIANI, Bagnolo in Piano (Reggio Emilia) (IT)

(73) Assignee: CTE INTERNATIONAL S.R.L., Reggio Emilia (IT)

(21) Appl. No.: 13/165,632

(22) Filed: Jun. 21, 2011

(51) Int. Cl. A61F 11/14

(52) U.S. Cl. 2/209

(57) ABSTRACT

An ear muff is provided and includes a mounting having at least an end portion to which a support element for a loudspeaker is associated. The support element is configured as a single body with the end portion of the mounting.

### FIELD OF INVENTION

The invention relates to an ear muff, and in particular an ear muff having an integrated earphone.

### BACKGROUND

As is known, an ear muff is a winter garment destined to be worn to protect the ears from the cold.

Some types of ear muff are constituted exclusively by an annular band made of wool or another heat-insulating material, which envelops the user's head, such as to cover the ears and leave the upper part of the head uncovered.

Other types of ear muff have an arched open-ringed shape.

These ear muffs generally comprise a semi-rigid plastic mounting, which exhibits two end portions destined to be applied to the ears, and an intermediate connecting portion destined to pass over or behind the head of the user.

These ear muffs comprise a woollen covering, or a covering made of another insulating material, which covers at least the end portions of the mounting. In the second above category there exists an ear muff model which is equipped with a pair of small loudspeakers hidden inside the covering and each one positioned at a respective end portion of the mounting.

The loudspeakers are associated to a connector, which is destined to be connected to an audio device, for example a CD player or a multi-media reader, such that the sounds reproduced by the device are heard by the user through the ear muffs. From the constructional point of view, each loudspeaker is coupled to a support element which is realised separately from the other earmuff components and which is fixed to the end portion of the mounting via appropriate connecting systems, for example by jointing or gluing.

In this way, however, the support elements of the loudspeakers complicate the realisation and mounting of the ear muffs, making them relatively slow and expensive to produce.

### SUMMARY

An aim of the present invention is to resolve or at least reduce the above drawback in the prior art.

A further aim of the invention is to attain the aforementioned objective in the ambit of a simple, rational and relatively inexpensive solution.

These aims are attained by the characteristics of the invention as reported in independent claim 1. The dependent claims delineate preferred and/or particularly advantageous aspects of the invention.

In particular, the invention makes available an ear muff comprising a mounting having at least an end portion to which is associated a support element for a loudspeaker, in which the support element is realised in a single body with the end portion of the mounting, for example by means of a single injection-moulding process of plastic material.

In this way, the number of components forming the ear muffs is advantageously reduced, making the mounting thereof simpler, more rapid and more economical.

In a preferred embodiment of the ear muffs, the support element comprises a small dish and a plurality of teeth which project from the dish, such as to define a claw destined to grip and fasten the loudspeaker.

Thanks to this solution, the fastening of the loudspeaker to the relative support element is substantially done by jointing, in a relatively simple and rapid way. In a preferred aspect of this embodiment, the support element further comprises a connecting portion destined to join the dish to the end portion of the mounting.

Obviously the connecting portion is realised in a single body both with the dish and the end portion of the mounting.

Thanks to this connecting portion, the support element of the loudspeaker can be positioned such as to be normally in the centre of the user's ear, guaranteeing a better listening quality.

The connecting portion is preferably of such a size as to be flexible, thus enabling a relative displacing of the support element with respect to the end portion of the mounting.

In this way, the ear muff is more comfortable for the user.

In a further preferred embodiment of the ear muff, the mounting comprises a connecting arch, to which the end portion is slidably coupled, such as to be able to slide between a retracted position and an advanced position, changing the overall length of the ear muffs, in order to adapt it to the head sizes of the various users.

In this context, the support element of the loudspeaker preferably comprises a recess destined to receive the terminal tract of the connecting arch, when the end portion is in a retracted position.

Thanks to this solution, when the end portion is in the retracted position, the overall dimensions of the ear muffs are extremely contained.

In a further preferred embodiment of the ear muff, the end portion of the mounting has an annular conformation which defines a fitting ring, and the support element for the loudspeaker is positioned internally of the ring.

In this way, the loudspeaker is always positioned substantially at the centre of the user's ear.

In a further preferred embodiment of the ear muff, supporting means of a device are also associated to the support element for the loudspeaker, which device is typically of an electronic type, destined to generate sound signals via the loudspeaker. Thanks to this solution, the electronic device remains associated to the ear muff, in an easy position for use and control thereof.

The electronic device can be a transceiver device, for example a Bluetooth technology device, destined to interact with other like devices, in order to enable communication between two or more people, or with other electronic devices, such as for example mobile telephones or multimedia readers.

In a preferred aspect of this embodiment, a heat-insulating cladding is interposed between the support element of the loudspeaker and the support means of the electronic device, which cladding covers the end portion of the mounting and the support element of the loudspeaker.

In this way, the loudspeaker remains hidden by the cladding, while the electronic device remains external, where it can be easily reached by the user.

In a further preferred aspect of the embodiment, the support means are conformed in such a way as to be able to releasably couple with the electronic device.

Thanks to this solution, the electronic device can be unhooked and rehooked as desired to and from the ear muff, for example in order for it to be used in other applications.

In a preferred embodiment, the ear muff comprises connecting means, for example an electric cable having a relative connector, destined to connect the loudspeaker associated to the support element with a device destined to generate sound signals via the loudspeaker.

The device can be the above-mentioned transceiver, or a radio, a CD reader or a multimedia reader.

In a further preferred embodiment, the ear muff further comprises a microphone.

In this way, the microphone can be connected to a transceiver device, such as the one mentioned herein above, enabling the user to communicate actively with other persons at a distance.

### BRIEF DESCRIPTION OF THE DRAWINGS

Further characteristics and advantages of the invention will better emerge from a reading of the following description provided by way of non-limiting example, with the aid of the figures as illustrated in the accompanying tables of drawings.

FIG. 1 is a perspective view of a mounting for an ear muff of the invention.

FIG. 2 is a frontal view of FIG. 1.

FIG. 3 is a lateral view of FIG. 1.

FIG. 4 is a side view of FIG. 1 opposite the side of FIG. 3.

FIG. 5 is a frontal view of the mounting of FIG. 1, in which only the earpiece 3 is shown, in a retracted position.

FIG. 6 is the view of FIG. 5, with the earpiece 3 in an advanced position.

FIG. 7 is the view indicated by the arrow VII of FIG. 6.

FIG. 8 is the view of FIG. 2, in which the mounting cladding is shown with a broken line.

FIG. 9 shows a transceiver device destined to be coupled to the ear muff of FIG. 8.

FIG. 10 is a perspective view of the transceiver device of FIG. 9.

FIGS. 11 and 12 shown the mounting of the transceiver device of FIG. 9 on the earpiece 3 of the ear muff.

### DETAILED DESCRIPTION OF THE PREFERRED EMBODIMENTS

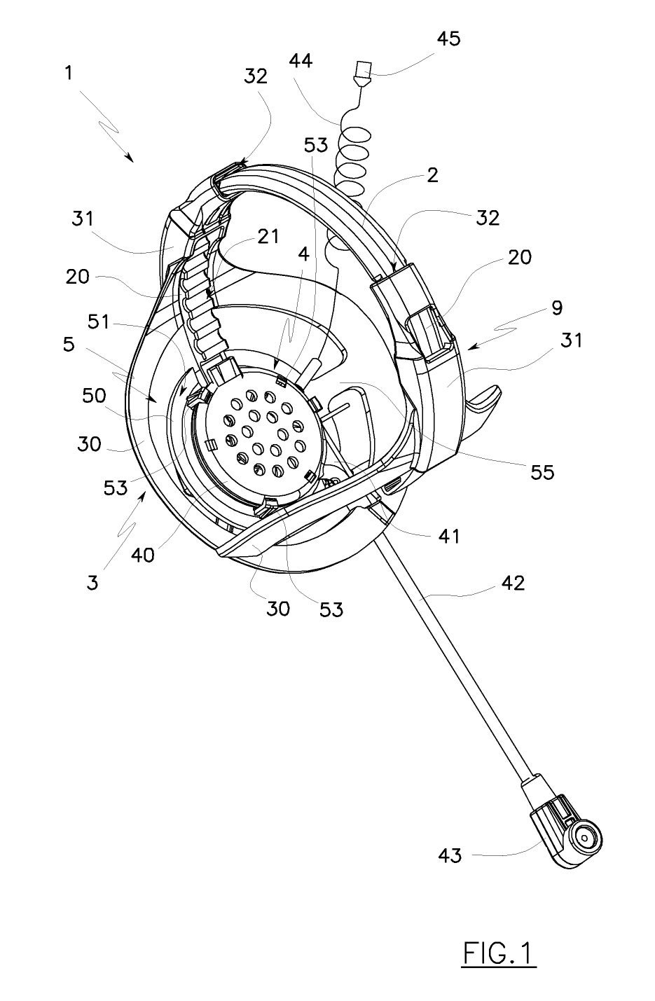

FIG. 1 shows a mounting 1 for ear muffs which comprises a flexible connecting arch 2, to opposite ends of which two earpiece shells are associated, indicated respectively by 3 and 9.

The flexible arch 2 and the two earpiece shells 3 and 9 are preferably made of plastic.

The earpiece shells 3 and 9 are destined to be applied to a user's ears, while the flexible arch 2 is destined to pass behind the head, partially enveloping it. Each earpiece shell 3 and 9 comprises a broadening 30, substantially formed as a slightly concave plate, the concavity of which is facing towards the broadening 30 of the opposite earpiece shell.

The broadening 30 has comparable dimensions, or is slightly bigger than a human ear.

The broadening 30 exhibits a large central opening, which gives it an annular conformation.

Each earpiece shell 3 and 9 further comprises an arched connecting spur 31, which is joined to the relative broadening 30, and exhibits a longitudinal guide passage 32 which inserts on a respective end tract 20 of the connecting arch 2.

Thanks to the coupling, each earpiece shell 3 and 9 can slide on a respective end tract 20 of the connecting arch 2 between a retracted position, in which the end tract 20 is deeply inserted on the spur 31, and an advanced position, in which the end tract 20 is partially disinserted from the spur 31.

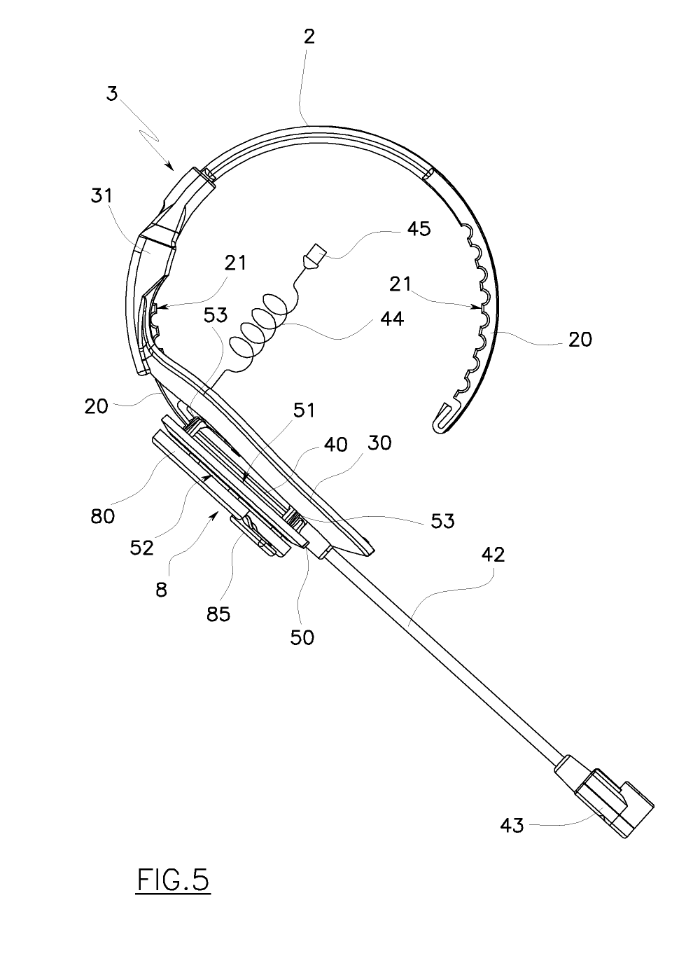

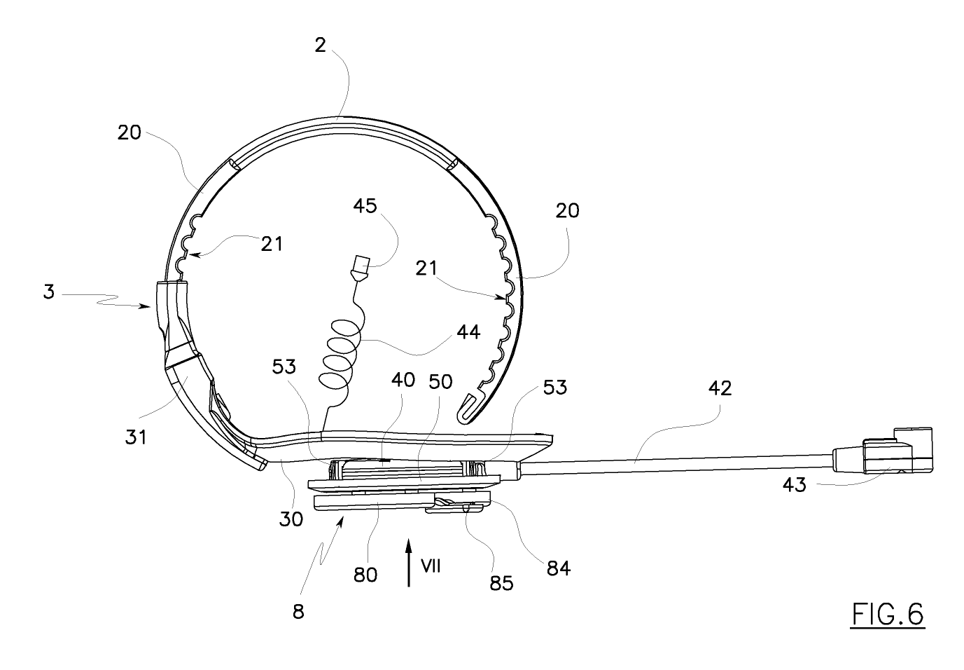

By way of example, FIGS. 5 and 6 illustrate the ear piece 3 respectively in the retreated position and the advanced position; the same positions being possibly attainable also independently by the earpiece shell 9 on the respective end tract 20.

Both the end tracts 20 exhibit a cogging 21, which is destined to cooperate with a tooth (not visible) afforded in the spur 31 of the relative earpiece shell 3 and 9. The tooth is destined to engage by a clicking mechanism in the cavities defined by the cogging 21, such as to be able to block the relative earpiece shell 3 and 9 in a plurality of intermediate positions between the retracted position and the advanced position thereof.

In this way, the length of the connecting arch 2 separating the earpiece shells 3 and 9 can be adjusted, such as to adapt the dimensions of the mounting 1 to those of the heads of the various users.

As illustrated in FIG. 1, the earpiece shell 3 comprises a support element 5 for a small loudspeaker 4.

The loudspeaker 4 comprises an external casing 40 of a substantially disc shape, which contains at least a loudspeaker (not visible).

In the illustrated embodiment, the external casing 40 exhibits an appendage 41, in which a hollow support arm 42 is supported, the support arm 42 bearing a microphone 43 at a free end thereof.

The microphone 43 is connected, via a wire running internally of the support arm 42, to a small electronic board (not visible) which is internal of the casing 40 and which is also connected to the loudspeaker.

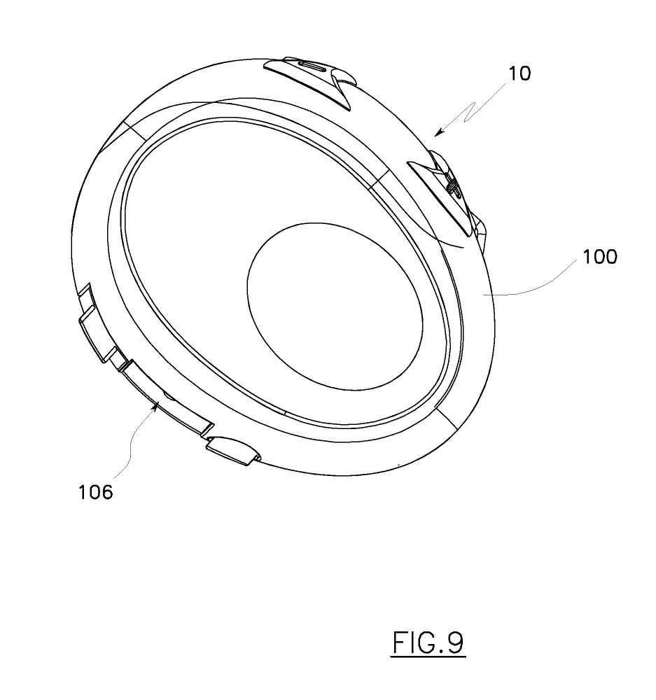

An electric connecting wire leaves the electronic board 44 and exits the casing 40 such as to bring a connector 45 to the outside, which connects the loudspeaker 4 to a transceiver device 10, illustrated in FIG. 9 and which will be more amply described herein below. The transceiver device 10 is destined to receive sound signals from the microphone 43 and to generate sound signals via the loudspeaker.

The support element 5 for the loudspeaker 4 comprises a dish 50 of a generally circular shape, which is located substantially coplanarly to the centre of the ring delimited by the broadening 30 of the earpiece shell 3.

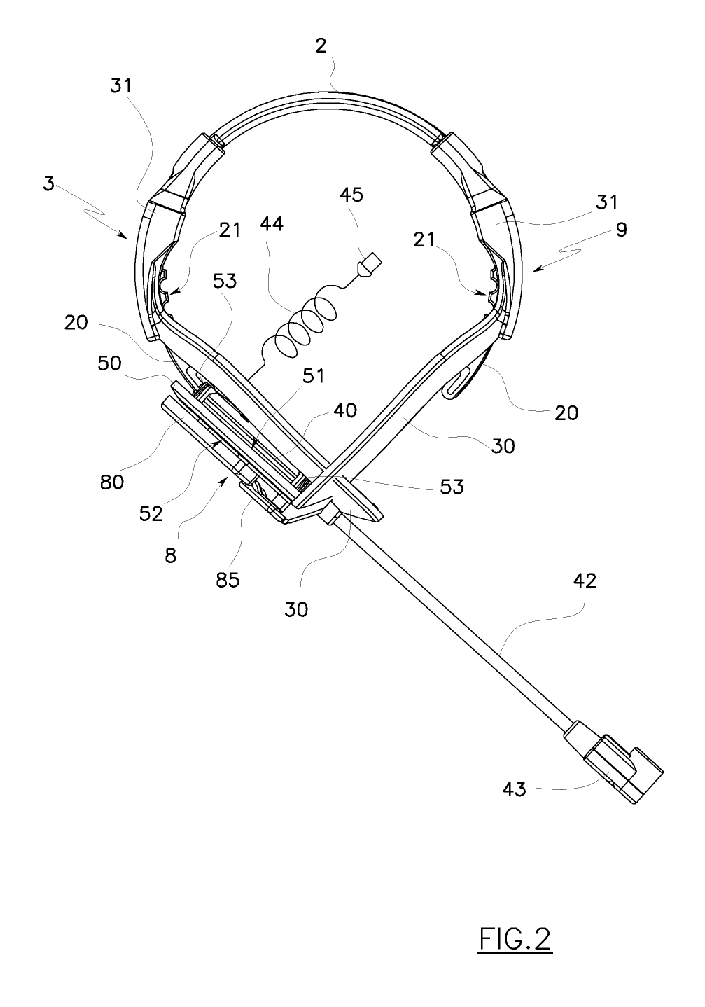

As illustrated in FIG. 2, the dish 50 exhibits an internal surface 51 facing towards the earpiece shell 9, and an opposite external surface 52.

Three teeth 53 project from the internal surface 51, which teeth 53 are ideally aligned along a common circumference, such as overall to define a claw which is destined to hook and retain the casing 40 of the loudspeaker 4.

In this way, the fixing of the loudspeaker 4 to the support element 5 is substantially done by jointing, in a relatively simple and rapid way.

Further, the loudspeaker 4 is positioned in the middle of the earpiece shell 3, such as to be positionable in the centre of the user's ear.

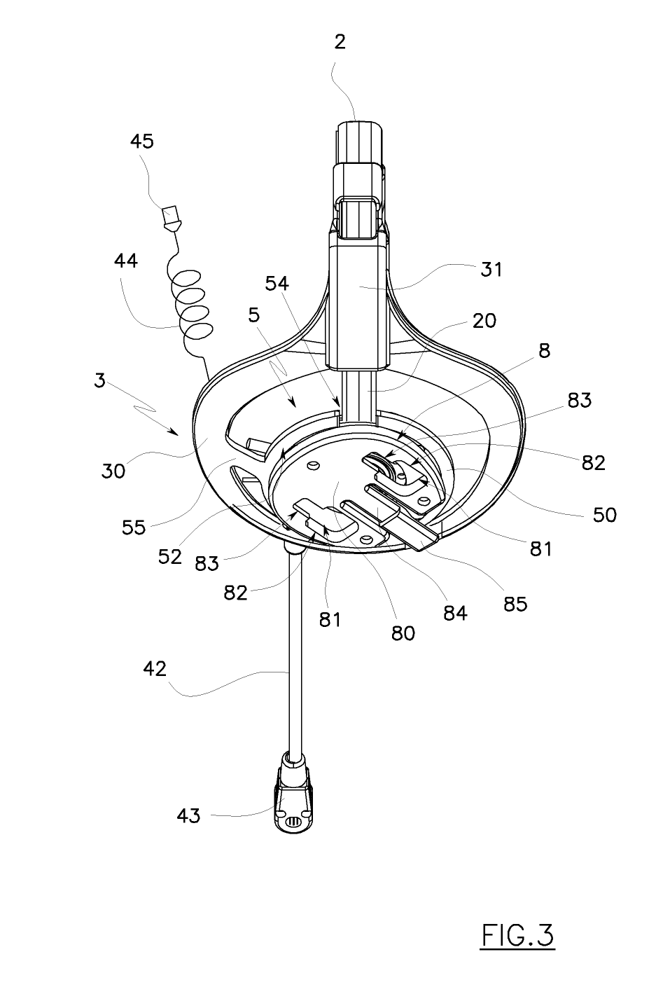

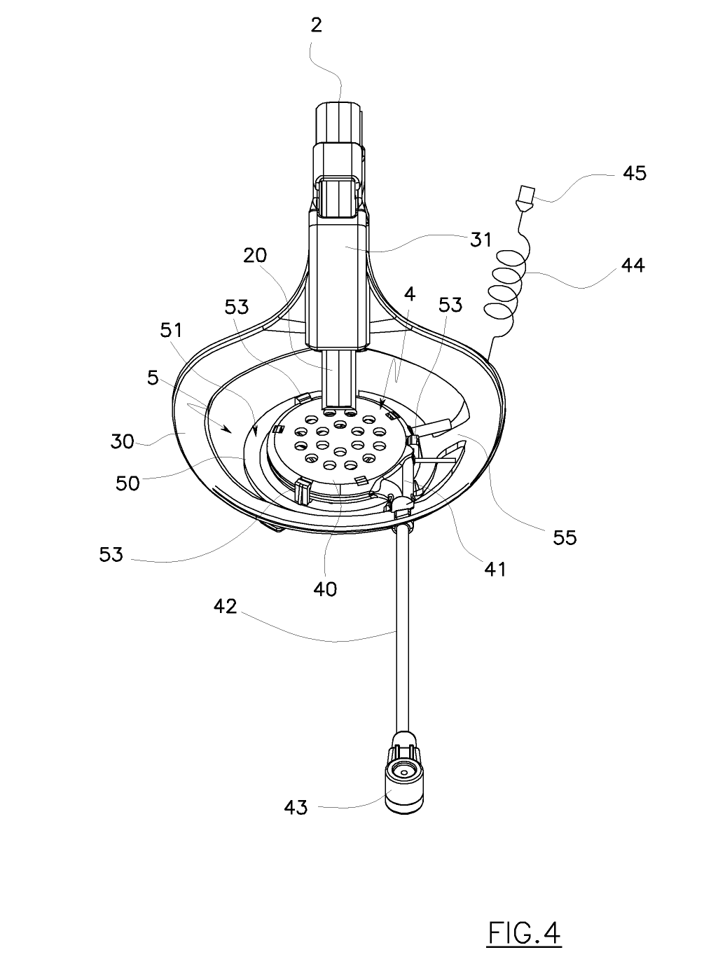

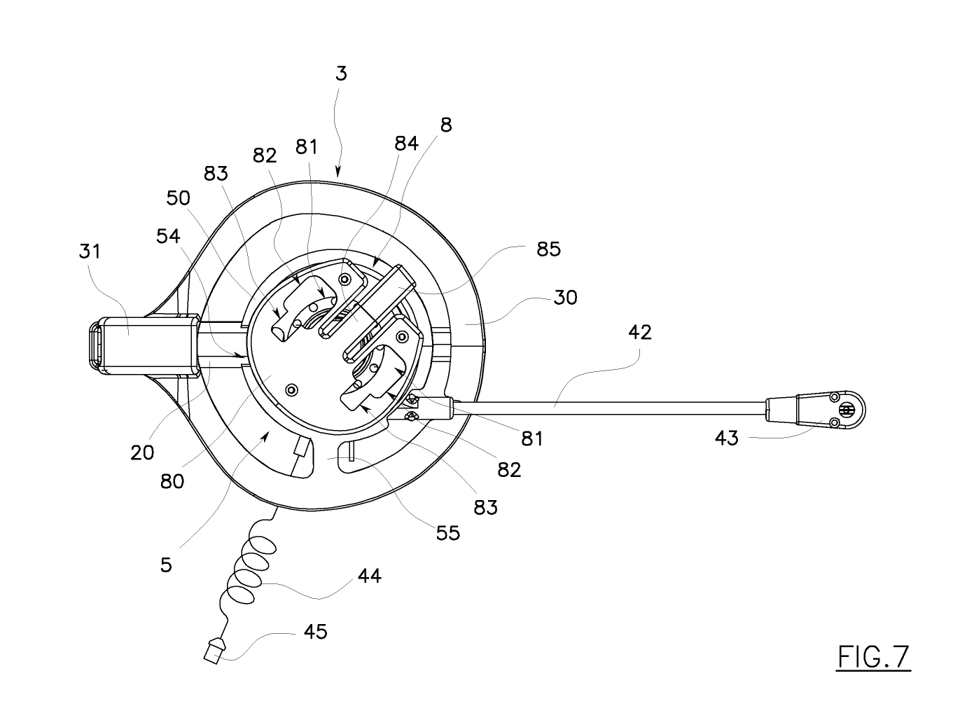

In order to reduce the size of the mounting 1 to a minimum, the perimeter profile of the dish 50 exhibits a recess 54 (see FIGS. 3 and 7), which is substantially aligned with the guide passage 32 of the spur 31, such as to be able to receive the terminal part of the end tract 20 of the connecting arch 2, when the earpiece shell 3 is in the retracted position.

The dish 50 is joined to the broadening 30 of the earpiece shell 3 via a single flat and narrow tongue 55 which is staggered by about 90° with respect to the recess 54.

In particular, the tongue 55 is of such dimensions as to be able to flex and thus enable small displacements of the dish 50 in a transversal direction with respect to the broadening 30, when the loudspeaker 4 is rested against the user's ear, which makes the ear muffs more comfortable.

In the illustrated example, the dish 50, the teeth 53 of the claw, the tongue 55, the broadening 30 and the spur 31 of the earpiece shell 3 are made in a single body, i.e. a monolithic body, for example by a single injection-moulding process of plastic materials.

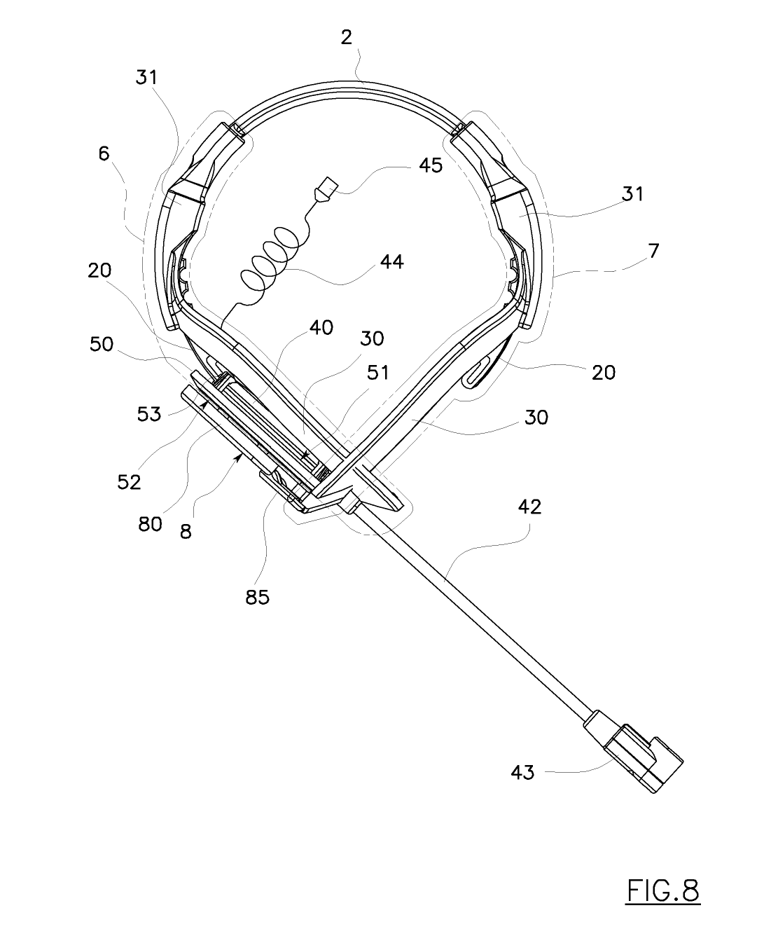

As is schematically illustrated in FIG. 8, the earpiece shell 3 is covered with a soft cover 6 made of a heat-insulating material, such as for example wool or fleece, which covers the broadening 30 on all sides, and closes over the connecting spur 31, such as to close the support element 5 and the loudspeaker 4 internally thereof.

The soft cover 6 obviously comprises an opening for enabling exit of the connecting wire 44 of the loudspeaker 4, and a hole for enabling the support arm 42 of the microphone 43 to exit, such that the microphone 43 can be neared to the user's mouth.

The earpiece shell 9 is in turn clad with a soft cover 7 made of a heat-insulating material, of the same type as the one used for the cover 6, which covers the broadening 30 on all sides and closes on the connecting spur 31.

In this way, the connecting arch 2 of the mounting 1 is partially uncovered.

A support base 8 for the transceiver device 10 is associated to the mounting 1 of the ear muff, which can be mounted on the ear muffs, in an appropriate position to be used and controlled.

The support base 8 is screwed to the external surface 52 of the dish 50, with the portion of the cover 6 covering it interposed.

In this way, the support base 8 is exposed externally of the cover 6, such that the transceiver device 10 can be easily mounted, dismounted and controlled. As illustrated in FIG. 3, the support base 8 comprises a dish 80 which is separated from the dish 50 by a slim space.

The plate 80 comprises two parallel slots 81, each of which comprises a wide portion 82 and a narrow portion 83.

Between the slots 81, the dish 80 is conformed such as to fashion a flexible tongue 84, the free end of which exhibits a projecting tooth 85.

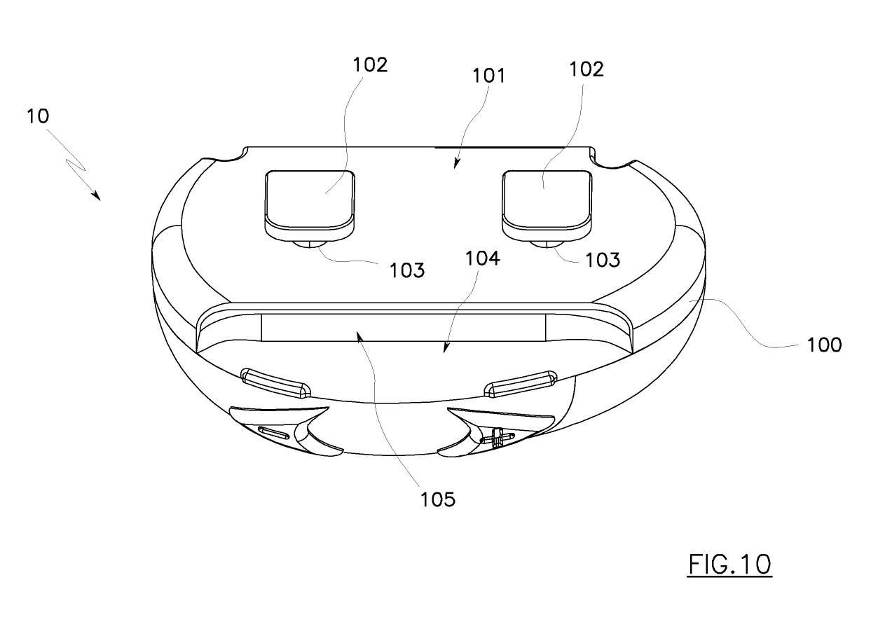

As illustrated in FIGS. 9 and 10, the transceiver device 10 comprises an external casing 10 having an oval shape, which contains the various electronic components of the device.

The casing 100 exhibits an internal surface 101 on which two skates 102 are afforded, which skates 102 have substantially the same shape in plan view as the wide portions 82 of the slots 81 of the support base 8.

The dishes 102 are distanced from the internal surface 101 by a respective projecting stalk 103, the transversal section of which substantially exhibits a same width as the narrow portions 83 of the slots 81.

A lowering 104 is further realised on the lower surface 101 which defines a step 105.

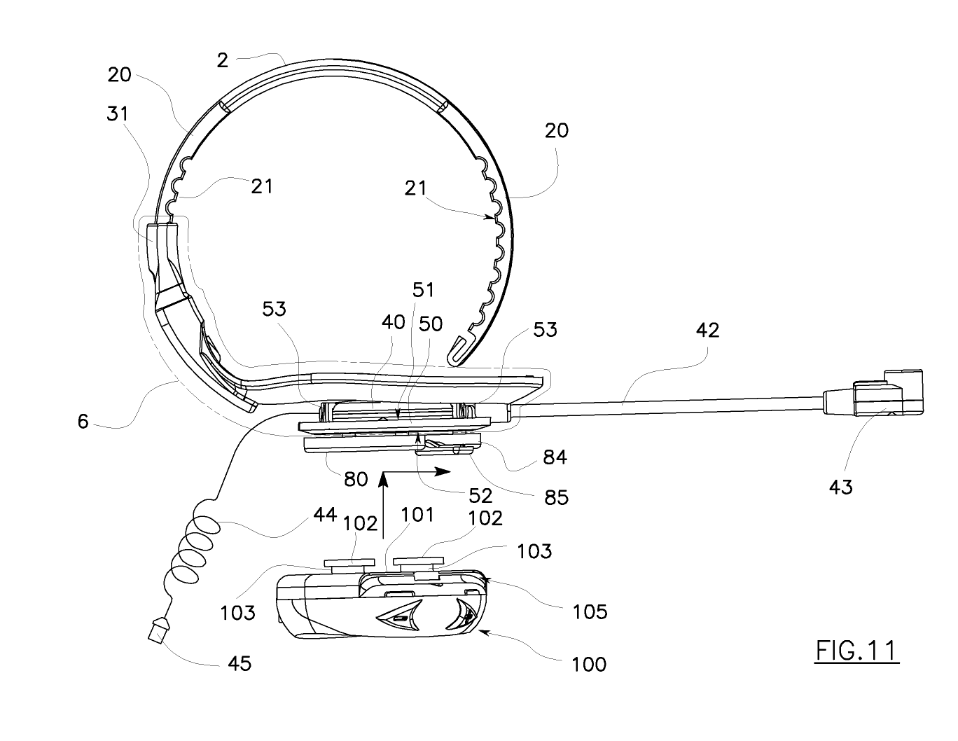

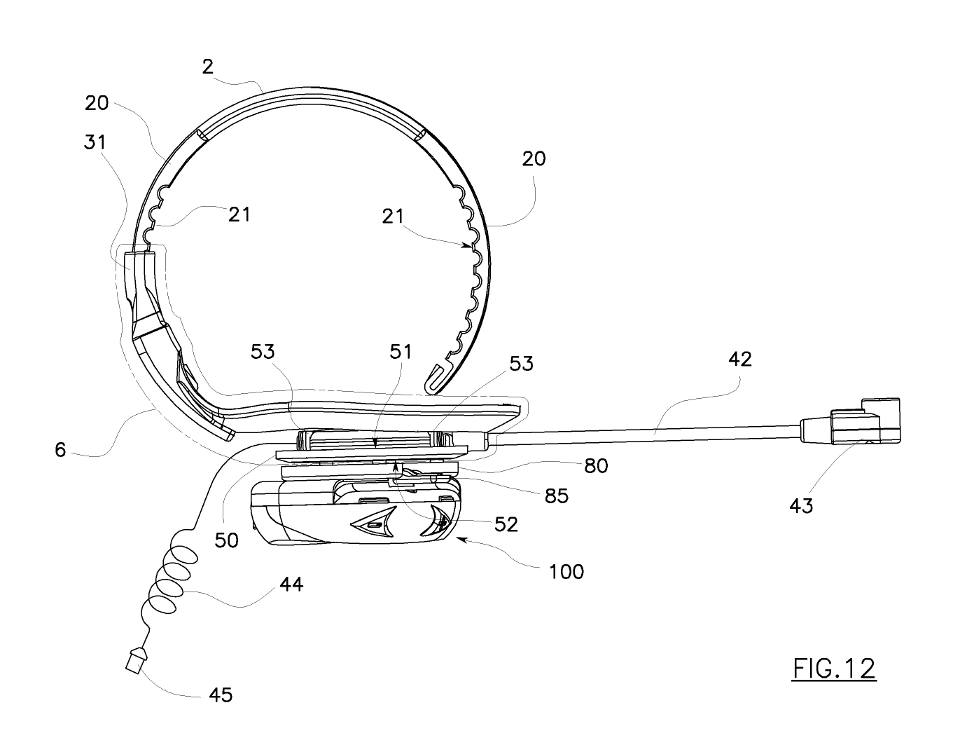

With reference to FIGS. 11 and 12, the coupling of the transceiver device 10 to the support base 8 is done as follows.

The skates 102 are inserted in the wide portions 82 of the slots 81, bringing the internal surface 101 of the casing 100 into contact with the projecting tooth 85 of the flexible tongue 84.

The casing 100 is thus pressed towards the dish 80, such as to flex the tongue 84, and then it is slid laterally such as to engage the stalks 103 of the skates 102 in the narrow portions 83 of the slots 81.

In this way, the skates 102 inserted below the dish 80, creating a snug fit constraint which prevents the casing 100 from separating from the support base 8. When the stalks 103 are completely inserted in the narrow portions 83 of the slots 81, the step 105 passes the projecting tooth 85 of the flexible tongue 84, which click-returns into the normal position thereof.

In this way, the tooth 85 faces the step 105, realising a snug fit constraint which prevents detaching of the stalks 103 from the narrow portions 83 of the slots 81. The dismounting of the transceiver 10 is done simply by deliberately flexing the tongue 84 and performing the above-described steps in reverse order.

The transceiver device 10 comprises a connecting port 106 (see FIG. 9) destined to receive the connector 45 borne by the connecting wire 44, such as to connect the transceiver device 10 to the loudspeaker 4.

In the illustrated embodiment, the transceiver device 10 uses Bluetooth technology, in order to be able to interact with a like transceiver device mounted for example on another earpiece, with the aim of enabling respective users to communicate to one another at distance.

The transceiver device 10 is therefore able to receive sound signals from the microphone 43 to transmit them to the other device, and is also able to receive sound signals from the other device in order to transform them into sounds via the loudspeaker 4 of the acoustic device.

In addition, or alternatively, the transceiver device 10 can interact with various electronic devices, such as for example mobile telephones or multi-media readers.

Although a transceiver device has been described herein, the acoustic device might be directly connected via the connecting wire 44 and a connector 45 also to an electronic device destined to generate only sounds, for example a radio, a CD reader or a multimedia reader.

In this case the electronic device would be separate from the ear muffs, and might thus be without the support base 8.

Obviously a technical expert might make numerous modifications of a technical-applicational nature to the earmuff described herein, without its thus forsaking the ambit of the invention as claimed herein below.

1). An ear muff comprising a mounting (1) having at least an end portion (3) to which a support element (5) for a loudspeaker (4) is associated, wherein the support element (5) is configured as a single body with the end portion (3) of the mounting (1).

2). The ear muff of claim 1, wherein the support element (5) comprises a dish (50) and a plurality of teeth (53) which project from the dish (50) in such a way as to define a claw configured to fasten the loudspeaker (4).

3). The ear muff of claim 2, wherein the support element (5) further comprises a connecting portion (55) configured to join the dish (50) to the end portion (3) of the mounting (1).

4). The ear muff of claim 3, wherein the connecting portion (55) is of such dimensions as to enable a relative displacement of the support element (5) with respect to the end portion (3) of the mounting (1).

5). The ear muff of claim 1, wherein the mounting (1) comprises a connecting arch (2), to which the end portion (3) is slidably coupled, such as to be able to slide between a retracted position and an advanced position thereof, and the support element (5) comprises a recess (54) configured to receive an end tract of the connecting arch (2), when the end portion (3) is in the retracted position.

6). The ear muff of claim 1, wherein the end portion (3) of the mounting (1) has an annular conformation which defines a ring, and the support element (5) for the loudspeaker (4) is positioned internally of the ring.

7). The ear muff of claim 1, wherein a support (8) is associated to the support element (5), said support (8) being for a device (10) configured to generate sound signals via the loudspeaker (4).

8). The ear muff of claim 7, wherein a heat-insulating covering (6) is interposed between the support element (5) and the support (8), said heat-insulating covering (6) clads the end portion (3) and the support element (5).

9). The ear muff of claim 7, wherein the support (8) is conformed such as to be dismountably couplable with the device (10).

10). The ear muff of claim 1, wherein it comprises a loudspeaker (4) associated to the support element (5).

11). The ear muff of claim 10, wherein it comprises a connector (44, 45) configured to connect the loudspeaker (4) with a device (10) configured to generate sound signals via the said loudspeaker (4).

12). The ear muff of claim 10, wherein it comprises a device (10) configured to generate sound signals via the loudspeaker (4).

13). The ear muff of claim 1 further comprising a microphone (43).
